(19) United States
(12) Patent Application Publication
(10) Pub. No.: US 2014/0276771 A1
(43) Pub. Date: Sep. 18, 2014

(54) SYSTEMS AND METHODS FOR CONTROLLED TISSUE ABLATION

(71) Applicant: VOLCANO CORPORATION, San Diego, CA (US)

(72) Inventors: David G. Miller, North Andover, MA (US); John W. Goodnow, Arlington, MA (US)

(73) Assignee: VOLCANO CORPORATION, San Diego, CA (US)

(21) Appl. No.: 14/211,039

(22) Filed: Mar. 14, 2014

Related U.S. Application Data
(60) Provisional application No. 61/792,847, filed on Mar. 15, 2013.

(51) Int. Cl. A61B 18/12

(52) U.S. Cl. CPC A61B 18/1233
USPC 606/34

(57) ABSTRACT

The invention describes methods for safely delivering ablation energy to a tissue, e.g., thrombus, in need of ablation therapy. The method uses a catheter adapted for IVUS imaging, ablation, and impedance measurements to monitor the impedance of a tissue receiving ablation energy. In an embodiment, a user may view an IVUS image of the tissue with impedance measurements to determine if it is safe to deliver additional energy. In another embodiment, a processor is configured to determine if it is safe to deliver additional ablation energy based upon the impedance measurement.

### RELATED APPLICATION

This application claims priority to U.S. Provisional Application No. 61/792,847, filed Mar. 15, 2013, which is incorporated by reference in its entirety.

### FIELD OF THE INVENTION

The invention relates to ablation catheters and methods of imaging and assessing tissues before and after ablation. The invention also describes methods for controlling an amount of energy delivered to the tissue.

### BACKGROUND

Ablation procedures typically involve contacting a tissue with a hot tool, such as a catheter tip, wire, or fluid. The heating process typically kills the outermost layer of cells contacting the tool, and may also (intentionally or unintentionally) damage deeper layers of cells. Some ablation procedures use directed energy, e.g., lasers, microwaves, or radiofrequency (RF) waves, while others simply use metallic structures that are heated via resistive (Joule) heating.

Ablation is commonly used in a number of medical specialties, including cardiology, gynecology, nephrology, dermatology, and endovascular surgery. In particular, cardiologists and endovascular surgeons can use ablation catheters to clear thrombus (i.e., plaque blockages) from blood vessels and to modify the muscles of the heart without needing to create an open surgical field. Typically, an ablation catheter is delivered to a targeted tissue via the brachial or femoral artery, and the procedure is guided with fluoroscopy or another imaging modality. Ablation catheters used for cardiac/endovascular procedures are typically either steerable rotating catheters with an ablating tip, e.g., the BLAZER™ catheter sold by Boston Scientific, or expandable element ablation catheters, e.g., the ENLIGHTN™ catheter sold by St. Jude Medical. Contemporary ablation catheters typically rely on gated energy delivery to control the temperature of the tissue. That is, the devices are programmed to provide a predetermined amount of energy over a predetermined time based upon accumulated experience and animal/cadaver studies.

While the ablation procedures are well-received, there are often complications from the procedures due to over- or under-heating as well as the difficulty of evaluating the procedure in real time. In most instances, the complications are relatively minor. For example, under-heating a tissue during a procedure will merely require the procedure to be repeated. In other instances, however, over-heating can lead to weakened, perforated, or severed vessels or loss of heart functionality. Without active temperature monitoring and control, it is very difficult to know if the tissue was overheated during the procedure. Furthermore, because the tissue is difficult to visualize during the procedure, it is hard to know exactly which tissues were treated.

### SUMMARY

The invention is a system for safely ablating tissue. In an embodiment, the system comprises an ablation catheter having an ablation member and being capable of IVUS imaging. The system additionally includes an impedance sensor and a controller that takes measurements from the impedance sensor and determines whether the impedance value is in excess of a safe value and, thus, whether it is safe to continue ablating the tissue. In other embodiments, the system images the tissue before and during ablation and uses the images to determine the safe application of ablation energy. In other embodiments, the system evaluates the tissue prior to ablation with the impedance sensor and combines the impedance measurements with the images to direct a course of ablation treatment. In other embodiments, the system can be programmed to automatically perform the ablation based upon imaging and/or impedance data obtained prior to beginning the procedure. In some embodiments, the system additionally uses feedback from the impedance sensor to evaluate the progress of a programmed ablation.

Using the disclosed ablation catheters, systems, and methods for applying energy to tissues, it will be safer to ablate tissues, and it will be easier to verify that the tissues have been properly heated to achieve the desired results. The safety measures disclosed in the invention will additionally reduce complication rates during ablation procedures.

### BRIEF DESCRIPTION OF THE DRAWINGS

FIG. 1 is a generalized depiction of a rotatable ablation catheter;

FIG. 2 is a generalized depiction of an ablation catheter having an expandable element;

FIG. 3 shows a method for imaging and treating a tissue with the same catheter;

FIG. 4 shows a method for imaging and treating a tissue with the same catheter;

FIG. 5 depicts an embodiment of a system for ablation with a catheter having imaging and impedance measurement capabilities;

FIG. 6 is a block diagram of an exemplary system for receiving imaging and impedance data and displaying relevant structure and tissue information;

FIG. 7 is a block diagram of an exemplary system for receiving imaging and impedance data and displaying relevant structure and tissue information;

FIG. 8 shows an intravascular ultrasound (IVUS) image of a vessel prior to ablation;

FIG. 9 shows a schematic of a blood vessel having regions of different tissue;

FIG. 10 shows the schematic of FIG. 9 overlaid on the IVUS image of FIG. 8;

FIG. 11 shows a flowchart detailing a method for controlling the amount of energy delivered to a tissue during an ablation procedure;

FIG. 12 shows a programmed ablative procedure that will deliver a small amount of heating to a defined sector of a blood vessel;

FIG. 13 shows a programmed ablative procedure that will deliver a large amount of heating to a defined sector of a blood vessel.

### DETAILED DESCRIPTION

The invention provides improved ablation catheters and methods of using the catheters to safely deliver energy to tissues in need of treatment. In particular, the catheters of the invention allow active monitoring of tissue impedance and optionally use intravascular ultrasound (IVUS) to reduce the rate of errors in delivering ablative treatment. The system and methods can be used for a variety of ablative procedures, but are well-suited for endovascular and cardiac procedures.

The process of heating a tissue to treat a disorder is generally known as “ablation,” even when the tissue is not removed. When ablation techniques were first pioneered, they were truly ablative, in that layers of tissue were burned away with high temperature tools. It has since been discovered that many disorders can be treated by merely heating, but not necessarily removing the tissue, because the heating causes changes to the tissue, e.g., scarring, or destroys/diminishes vasculature or nerves underlying the tissue. For example, endometrial ablation is commonly used to control uterine bleeding. Endometrial ablation involves heating the tissue of the uterine lining to cause the tissue to scar and to dilate the underlying vasculature.

FIG. 1 shows an embodiment of a rotational ablation catheter having an ablation member 16, impedance sensors 18, an ultrasound transducer 35 and a steerable tip 28. In the embodiment shown in FIG.1, rotation and steering of the catheter can be controlled by the user by adjusting the manipulators 24 and 26 on the handle 12. Power and control for the ablation member 16, impedance sensor 18, and ultrasound transducer 18 is provided via pigtail connection 22. The pigtail connection 22 is typically interfaced to a controller, discussed in greater detail in the following figures. When in use the rotational ablation catheter is typically inserted into a lumen, i.e., an artery and directed to the tissue to ablated with steering and pushing. Other embodiments of a rotational ablation catheter may comprise mechanized drive cables (not shown) that allow the rotation of the ablation member 16 to be coordinated through a controller.

FIG. 2 shows an embodiment of a balloon ablation catheter system 10 for treating tissues with heat. The catheter system 10 includes a balloon catheter 12 having a catheter body 14 with a proximal end 16 and a distal end 18. Catheter body 14 is flexible and defines a catheter axis 15, and may include one or more lumens, such as a guidewire lumen and an inflation lumen. Additional lumens may be provided for other treatments, such as imaging, perfusion, fluid delivery, etc. Catheter 12 includes an inflatable balloon 20 adjacent distal end 18 and a housing 29 adjacent proximal end 16. When inflated and energized, inflatable balloon 20 provides thermal RF energy to the tissue, causing it to increase in temperature. Housing 29 includes a first connector 26 in communication with the guidewire lumen and a second connector 28 in fluid communication with the inflation lumen (not shown). The inflation lumen extends between balloon 20 and second connector 28. Both first and second connectors 26, 28 may optionally comprise standard connectors, such as Luer-Loc™ connectors.

Housing 29 also accommodates an electrical connector 38. Connector 38 includes a plurality of electrical connections, each electrically coupled to electrodes 34 via conductors (not shown). Electrodes 34 are energized and controlled by a controller 40 and power source 42, such as bipolar or monopolar RF energy, microwave energy, ultrasound energy, voltage source, current source, or other suitable energy source. In an embodiment, electrical connector 38 is coupled to an RF generator via a controller 40, with controller 40 allowing energy to be selectively directed to electrodes 38. When monopolar RF energy is employed, the patient may be grounded by connecting an external electrode, or an electrode connected to the catheter body 14, to the patient.

In the embodiment shown in FIG. 2, the controller 40 includes a processor, or is coupled to a processor, to control and/or record treatment. The processor will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of catheter system 10 and within processor 40.

Balloon 20 generally includes a proximal portion coupled to an inflation lumen and a distal portion coupled to a guidewire lumen. Balloon 20 expands radially when inflated with a fluid or a gas. Balloon 20 is constructed from a compliant material that can withstand heat and high pressures. Prior to inflation, balloon 20 is positioned in the distal end 18 of the catheter. Balloon 20 has helical folds to facilitate conversion between an expanded (inflated) configuration and a low profile configuration, needed for delivery and removal.

In an embodiment, the balloon 20 is configured with electrodes 34 integrated into the wall of the balloon 20 to deliver RF energy to heat tissues. The electrodes 34 may be mounted on an inside surface of balloon 20, with associated connectors/wires extending proximally from the electrodes. The electrodes 34 may be mounted on an inside surface of the balloon 20. The electrodes 34 may be sandwiched between layers of balloon material. The electrodes 34 may be arranged in any suitable pattern, such as stripes, helixes, saw tooth, rings, or arrays.

The system may be used for monopolar or bipolar application of energy. For delivery of monopolar energy, a ground electrode is used, either on the catheter shaft, or on the patient's skin, such as a ground electrode pad. For delivery of bipolar energy, adjacent electrodes are axially offset to allow bipolar energy to be directed between adjacent circumferential (axially offset) electrodes. In other embodiments, electrodes may be arranged in bands around the balloon to allow bipolar energy to be directed between adjacent distal and proximal electrodes.

In an embodiment, the system heats tissues using heated fluids. In this configuration, balloon 20 need not include electrodes 34. In this embodiment, the balloon is substantially impervious to aqueous solutions, e.g., saline, to prevent the heated fluid from leaving the balloon. In an embodiment, the catheter includes an insulated lumen for delivering heated fluids to the balloon, e.g., heated saline. The fluid may have a temperature of 37° C. or greater, e.g., 40° C. or greater, e.g., 45° C. or greater, e.g., 50° C. or greater, e.g., 55° C. or greater, e.g., 60° C. or greater, e.g., 65° C. or greater, e.g., about 68° C. Systems of the catheter 10, configured to heat tissues with heated fluids may comprise a heated fluid reservoir and a pump connected to inflation lumen to deliver the heated fluids (not shown). Other embodiments for heating tissues with heated fluids may comprise a heating element inside of the balloon as an element of the catheter. The balloon may be filled in the traditional method, i.e., with room or body temperature saline directed to the balloon via an inflation lumen, and then the fluid can be heated with the heating element to provide a heated fluid. In some embodiments, a balloon catheter will also include a temperature sensor located proximate to the center of the balloon to be used to measure the temperature of the heated fluid.

In an embodiment, the balloon 20 is configured with temperature sensors integrated into the wall of the balloon. The temperature sensors may be mounted on an inside surface of balloon 20, with associated connectors/wires extending proximally from the temperature sensors. The temperature sensors may be mounted on an inside surface of the balloon 20. The temperature sensors may be sandwiched between layers of balloon material. The temperature sensors may be arranged in any suitable pattern, such as an array. The temperature sensors may be any temperature sensor that has a sufficiently small profile to be incorporated into the balloon, for example the temperature sensors may be a thermocouple, thermistor, thermal diode, or other suitable device. In some embodiments, the catheter will comprise an additional heating element that is inside the balloon, e.g., in proximity to a distal end of the inflation lumen, thereby allowing the inflation fluid, e.g., a heated inflation fluid, to be monitored.

Methods of imaging and ablating with catheters are described in FIGS. 3 and 4. As shown in FIG. 3, the ablation catheter may comprise an ultrasound transducer and an ablative member. Optionally, as shown in FIG. 3, the catheter may comprise a stabilization balloon that facilitates stable rotation and assures that imaging and ablation are spatially overlapped. The catheter and method in FIG. 3 differ from catheter and method in FIG. 4 in that the catheter of FIG. 3 images and ablates radially whereas the catheter of FIG. 4 images and ablates in the forward direction. Other configurations may be used with the methods of the invention.

In a preferred embodiment, the ablation catheter comprises one or more ultrasound transducers and is configured to image the tissue with intravascular ultrasound (IVUS) techniques. IVUS catheters and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In other embodiments, different imaging modalities may be used, such as optical coherence tomography (OCT). OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

The ablation catheters are part of a system for evaluating tissues and safely delivering ablation energy to the tissues. An exemplary system 500 is shown in FIG. 5. The system includes a catheter 100 having an ablation member, an impedance sensor, and an ultrasound transducer. The system additionally includes a subcontroller for each function, e.g., IVUS controller 340, Ablation Controller 440, and Impedance Controller 540. Each subcontroller is operatively connected to the system controller 640 that coordinates all of the functionality. The system controller 640 also synchronizes the functionality of each aspect of the system.

As shown in FIG. 5, the system controller is interfaced to image processing 360 that is capable of synthesizing the images and tissue measurements into easy-to-understand images. As discussed in greater detail below, the image processing will deconvolve the reflected acoustic wave to produce distance and/or tissue measurements, and those distance and tissue measurements can be used to produce an image, for example an intravascular ultrasound (IVUS) image. The image processing may additionally include spectral analysis, i.e., examining the energy of the returned acoustic signal at various frequencies. Spectral analysis is useful for determining the nature of the tissue and the presence of foreign objects. A plaque deposit, for example, will typically have a different spectral signature than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. Other signal processing techniques known in the art of tissue characterization may also be applied.

Other image processing may facilitate use of the images or identification of features of interest. For example, the border of a lumen may be highlighted or plaque deposits may be displayed in a visually different manner (e.g., by assigning plaque deposits a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. Other measurements, such as flow rates or pressure may be displayed using color mapping or by displaying numerical values.

A system of the invention may be implemented with a variety of architectures. An embodiment of a system 300 of the invention is shown in FIG. 6. The core of the system 300 is a computer 360 or other computational arrangement comprising a processor 365 and memory 367. The memory has instructions which when executed cause the processor to determine a baseline measurement prior to conducting a therapeutic procedure and determine a post-therapy measurement after conducting the therapeutic procedure. The instructions may also cause the computer to compare the post-therapy measurement to the baseline measurement, thereby determining the degree of post-therapy improvement after conducting the therapeutic procedure. In the system of the invention, the physiological measurement data of vasculature will originate with a catheter 100 as discussed above, whose function is controlled with a system controller 640. Having collected the image data, the processor then processes the data to build images and identify flow and/or structures and then outputs the results. The results are typically output to a display 380 to be viewed by a physician or technician.

In advanced embodiments, system 300 may comprise an imaging engine 370 which has advanced image processing features, such as image tagging, that allow the system 300 to more efficiently process and display intravascular and angiographic images. The imaging engine 370 may automatically highlight or otherwise denote areas of interest in the vasculature. The imaging engine 370 may also produce 3D renderings or other visual representations of the physiological measurements. In some embodiments, the imaging engine 370 may additionally include data acquisition functionalities (DAQ) 375, which allow the imaging engine 370 to receive the physiological measurement data directly from the catheter 325 or collector 347 to be processed into images for display.

Other advanced embodiments use the I/O functionalities 362 of computer 360 to control the detector or to trigger the light source for the catheter. While not shown here, it is also possible that computer 360 may control a manipulator, e.g., a robotic manipulator, connected to catheter 325 to improve the placement of the catheter 100.

A system 400 of the invention may also be implemented across a number of independent platforms which communicate via a network 409, as shown in FIG. 7. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 7, the system controller 604 facilitates obtaining the data, however the actual implementation of the steps can be performed by multiple processors working in communication via the network 409, for example a local area network, a wireless network, or the internet. The components of system 400 may also be physically separated. For example, terminal 467 and display 380 may not be geographically located with the intravascular detection system 320.

As shown in FIG. 7, imaging engine 859 communicates with host workstation 433 as well as optionally server 413 over network 409. In some embodiments, an operator uses host workstation 433, computer 449, or terminal 467 to control system 400 or to receive images. An image may be displayed using an I/O 454, 437, or 471, which may include a monitor. Any I/O may include a monitor, keyboard, mouse, or touch screen to communicate with any of processor 421, 459, 441, or 475, for example, to cause data to be stored in any tangible, nontransitory memory 463, 445, 479, or 429. Server 413 generally includes an interface module 425 to communicate over network 409 or write data to data file 417. Input from a user is received by a processor in an electronic device such as, for example, host workstation 433, server 413, or computer 449. In certain embodiments, host workstation 433 and imaging engine 855 are included in a bedside console unit to operate system 400.

In some embodiments, the system may render three dimensional imaging of the vasculature or the intravascular images. An electronic apparatus within the system (e.g., PC, dedicated hardware, or firmware) such as the host workstation 433 stores the three dimensional image in a tangible, non-transitory memory and renders an image of the 3D tissues on the display 380. In some embodiments, the 3D images will be coded for faster viewing. In certain embodiments, systems of the invention render a GUI with elements or controls to allow an operator to interact with three dimensional data set as a three dimensional view. For example, an operator may cause a video affect to be viewed in, for example, a tomographic view, creating a visual effect of travelling through a lumen of vessel (i.e., a dynamic progress view). In other embodiments an operator may select points from within one of the images or the three dimensional data set by choosing start and stop points while a dynamic progress view is displayed in display. In other embodiments, a user may cause an imaging catheter to be relocated to a new position in the body by interacting with the image.

In some embodiments, a user interacts with a visual interface and puts in parameters or makes a selection. Input from a user (e.g., parameters or a selection) are received by a processor in an electronic device such as, for example, host workstation 433, server 413, or computer 449. The selection can be rendered into a visible display. In some embodiments, an operator uses host workstation 433, computer 449, or terminal 467 to control system 400 or to receive images. An image may be displayed using an I/O 454, 437, or 471, which may include a monitor. Any I/O may include a keyboard, mouse or touch screen to communicate with any of processor 421, 459, 441, or 475, for example, to cause data to be stored in any tangible, nontransitory memory 463, 445, 479, or 429. Server 413 generally includes an interface module 425 to effectuate communication over network 409 or write data to data file 417. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 433 and imaging engine 855 are included in a bedside console unit to operate system 400.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 380 is rendered within a computer operating system environment, such as Windows, Mac OS, or Linux or within a display or GUI of a specialized system. Display 380 can include any standard controls associated with a display (e.g., within a windowing environment) including minimize and close buttons, scroll bars, menus, and window resizing controls. Elements of display 380 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 380 can further include any controls or information related to viewing images (e.g., zoom, color controls, brightness/contrast) or handling files comprising three-dimensional image data (e.g., open, save, close, select, cut, delete, etc.). Further, display 380 can include controls (e.g., buttons, sliders, tabs, switches) related to operating a three dimensional image capture system (e.g., go, stop, pause, power up, power down).

In certain embodiments, display 380 includes controls related to three dimensional imaging systems that are operable with different imaging modalities. For example, display 380 may include start, stop, zoom, save, etc., buttons, and be rendered by a computer program that interoperates with IVUS, OCT, or angiogram modalities. Thus display 380 can display an image derived from a three-dimensional data set with or without regard to the imaging mode of the system.

Using the system of the invention, it is possible to image and treat tissue simultaneously. As shown in FIG. 8, the tissue, e.g., a vessel can be imaged and evaluated for condition of the tissue. The system can determine, e.g., the placement of plaque of other materials based upon imaging and impedance measurements. Once evaluated, the system can construct a variety of images to communicate the condition of the tissue to the user. For example, as shown in FIG. 9, a simple image can be constructed with shapes and colors indicating the position of different materials. For example, as shown in FIG. 9, the lower left quadrant may have sclerotic plaque, while the lower right quadrant has a bulging arterial wall. Alternatively, the information can be superimposed on the image of the vessel, e.g., as shown in FIG. 10. Such a display will make it easy for the surgeon to identify how the ablation is performed, and may be used to direct auto-ablation, as discussed below.

Using the systems of the invention, it will not be necessary to gate the energy delivery during treatment. Rather, it will be a simple matter of comparing the impedance measured with a predetermined impedance. This method is shown in greater detail in FIG. 11. As discussed above with respect to FIGS. 1-4, the method begins with placing the ablation member near the tissue to be treated. Impedance sensors monitor the impedance of the tissue being treated to determine a measured impedance (Iₘ). Iₘ is then compared to a predetermined impedance for treatment, I_{MAX}, or critical impedance. If Iₘ is less than I_{MAX}, the catheter is allowed to continue delivering therapy via the heating elements 320. However if Iₘ is equal to or greater than I_{MAX}, the ablative member is de-energized, to avoid damaging the tissue. Once the therapy is completed, the tissue may be reassessed with imaging and impedance measurements.

Advanced embodiments of the methods may include algorithms for monitoring or measuring the treatment area impedance. For example, readings from multiple impedance sensors 330 at different points on expandable member 330 may be modeled to develop a heat map of how the tissue is heating. Additionally, if the ablation members are individually addressable, it may be possible to turn some off and leave others on in order to achieve more even heating. Other algorithms may be used to estimate overshoot to determine if and when the heating elements should be turned off prior to Iₘ exceeding I_{MAX}.

An additional feature of the invention is using the information obtained with the imaging and/or impedance measurements to control the ablative process. The invention thus allows the position and the strength of the ablation to be programmed and executed. Using the disclosed system, it is also possible to display and program treatment using the graphical user interface described above.

For example, in an embodiment, after evaluating a vessel, a physician would like to direct ablation limited to a segment of the interior of a vessel. It is based upon the value of the electrical impedance along a small area of the surface of the lumen. With the control system described above it is possible to automatically switch the ablative member on and off based upon the impedance measurement. This feature permits ablation over only a segment of the lumen rather than the entire internal diameter. This avoids ablating healthy tissue while plaque is being removed and it permits directing the catheter along the curvature of the vessel rather than plunging straight ahead. It is intended to be used along with IVUS for guidance and additional information. The catheter is employed in cases where the vessel has become restricted or entirely blocked by plaque.

When applying therapy to a vascular lesion it is also useful to automatically control the area that is being ablated rather than the entire volume of tissue in the immediate vicinity of the tip of the electrode. A preferred embodiment would use a rotating catheter with a mechanized rotating ablation member interfaced with the controller. The controller may also make use of the impedance sensor which is optionally in communication with a small electrode or ground plate on the surface of the patents body or a local ground on the catheter or a second electrode near the catheter tip.

This system is programmable to provide tissue ablation limited to a segment of the interior of a vessel. As discussed above with respect to FIGS. 8-10, an IVUS display (with or without the “true” image of the vessel) can be used to provide information on the location of the plaque and the location of the vessel wall. Next the system can be programmed to deliver treatment based upon the images, e.g., as shown in FIGS. 12 and 13. In FIGS. 12 and 13, sector outlines is superimposed on the display. The angular extent of the ablation is shown by the angular width of the display and the intensity of the ablation is indicated by the radial length of the sector outline. As an example, FIG. 12 may correspond to ablation set for a 90 sector at low power centered at an angular position of 345 degrees from the 12:00 position, while FIG. 13 corresponds to ablation set for a 150 degrees sector at high power centered at an angular position of 345 degrees from the 12:00 position. This feature permits ablation over only a segment of the lumen rather than the entire internal diameter. This avoids ablating healthy tissue while plaque is being removed and it permits directing the catheter along the curvature of the vessel rather than plunging straight ahead and possibly dissecting the vessel. The catheter is employed in cases where the vessel has become restricted or is entirely blocked by plaque.

Because the invention incorporates an electrode on the outside edge of an IVUS catheter that supplies electrical energy to ablate tissue. The electrical energy is switched on and off in phase with the rotation of the catheter such that only a segment of the tissue near the catheter electrode is ablated. This provides two benefits. First, only a segment of the plaque is removed. Second, tissue removal by this method permits the catheter to follow the curved contour and tortuosity of the vessel without perforation.

Other embodiments of catheters and methods of using them, not disclosed herein, will be evident to those of skill in the art, and are intended to be covered by the claims listed below.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for delivering energy to a tissue, comprising:
delivering to a tissue a catheter adapted for intravascular ultrasound (IVUS) imaging, impedance measurement, and tissue ablation;
imaging the tissue with an IVUS transducer;
measuring a first impedance of the tissue with an impedance sensor; and
ablating the tissue with an ablation member.

2. The method of claim 1, further comprising comparing the first impedance to a predetermined impedance and determining if additional ablation may be safely delivered to the tissue.

3. The method of claim 1, further comprising measuring a second impedance of the tissue, comparing the second impedance to a predetermined impedance, and determining if additional ablation may be safely delivered to the tissue.

4. The method of claim 1, further comprising measuring a second impedance of the tissue, comparing the first and second impedances, and determining if additional ablation may be safely delivered to the tissue.

5. The method of claim 1, wherein the ablation member is an ablation electrode located at a distal end of the catheter.

6. The method of claim 1, wherein the ablation member is expandable.

7. The method of claim 6, wherein the expandable ablation member is a balloon.

8. The method of claim 1, wherein the catheter comprises a plurality of electrodes.

9. The method of claim 8, wherein the electrodes provide energy to the tissue and allow impendence measurements.

10. The method of claim 1, wherein the tissue is cardiac tissue or vascular tissue.

11. The method of claim 1, wherein the catheter comprises a location indicator.

12. A system for ablating a tissue, comprising:
a catheter comprising an intravascular ultrasound (IVUS) transducer, an impedance sensor, and an ablation member; and
a controller comprising a processor and memory, operatively connected to the impedance sensor and the ablation member, wherein the memory comprises instructions that when executed cause the processor to:
receive an impedance measurement from the sensor,
compare the impedance measurement to a predetermined impedance value, and
allow operation of the ablation member based upon the comparison between the measured and predetermined impedance value.

13. The system of claim 12, further comprising an imaging engine capable of receiving ultrasound data and producing an image of the tissue.

14. The system of claim 13, wherein the imaging engine additionally receives values from the impedance sensor and produces an enhanced image of the tissue including impendence measurements.

15. The system of claim 14, wherein the impendence measurements are differentiated with color.

16. The system of claim 14, further comprising a display, wherein the enhanced image of the tissue including impendence measurements is displayed on the display.

17. The system of claim 13, wherein the imaging engine additionally displays a second image of the tissue produced with fluoroscopy.

18. A system for ablating a portion of a tissue, comprising:
a catheter having a rotatable ablation member mechanically coupled to a rotational controller;
an ablation controller; and
a master controller comprising a processor and memory, operatively connected to the rotational controller and the ablation controller, wherein the memory comprises instructions that when executed cause the processor to coordinate rotation of the ablation member with energy delivery by the ablation member such that only a portion of the tissue receives ablation energy.

19. The system of claim 18, further comprising an impedance sensor operably coupled to the master controller, and wherein the memory additionally comprises instructions that when executed cause the processor to receive an impedance measurement from the sensor, compare the impedance measurement to a predetermined impedance value, and allow operation of the ablation member based upon the comparison between the measured and predetermined impedance value.

20. The system of claim 19, wherein the catheter additionally comprises a location indicator and the system additionally comprises a location receiver configured to receive location information about the catheter from outside of a patient undergoing an ablation procedure.
